(19) United States
(12) Patent Application Publication
(10) Pub. No.: US 2010/0183557 A1
(43) Pub. Date: Jul. 22, 2010

(54) PTRA GENE AND USES THEREOF

(75) Inventors: Teresa De Kievit, Headingly (CA); Dilantha Fernando, Winnipeg (CA)

Correspondence Address: GOWLING LAFLEUR HENDERSON LLP, P.O. BOX 30 SUITE 2300 - 550 Burrard Street, VANCOUVER - BRITISH COLUMBIA, V7X 1J1, CA

(73) Assignee: UNIVERSITY OF MANITOBA, Winnipeg, MB (CA)

(21) Appl. No.: 12/446,745

(22) PCT Filed: Oct. 24, 2007
(86) PCT No.: PCT/CA2007/001913

§ 371 Date: Feb. 1, 2010

Related U.S. Application Data
(60) Provisional application No. 60/853,752, filed on Oct. 24, 2006.

(51) Int. Cl. A61K 35/74; C07H 21/04; C07K 14/21; C12N 15/63; C12N 1/00; C12N 1/21; A61P 31/04

(52) U.S. Cl. 424/93.2; 536/23.1; 536/23.7; 530/350; 435/320.1; 435/243; 435/252.34

(57) ABSTRACT

An isolated nucleic acid molecule comprising a nucleotide sequence set forth in SEQ ID NO: 1, said nucleotide sequence encoding a protein inhibitory to Sclerotinia spp., said protein comprising an amino acid sequence set forth in SEQ ID NO: 2. A nucleotide construct comprising the nucleic acid molecule operably linked to a suitable promoter, may be stably incorporated into the genome of a selected cell for expression of the nucleotide sequence therein. Suitable cells are exemplified by Pseudomonas sp. Compositions comprising microbial cells comprising the nucleotide sequence set forth in SEQ ID NO: 1, may be applied to Brassica seeds or alternatively, to developing Brassica plants to inhibit Sclerotinia infections thereof and therein.

### FIELD OF THE INVENTION

This invention relates to biocontrol of plant pathogens. More particularly, this invention relates to regulation of microbial synthesis of metabolites that interfere with plant pathogen proliferation and/or their infection of plants.

### BACKGROUND OF THE INVENTION

It is well known that many Pseudomonas spp. are capable of suppressing the proliferation of plant pathogens on laboratory media and their in situ infection of plant hosts in controlled environmental conditions, and therefore, are commonly referred to as biocontrol organisms. Biocontrol organisms typically produce as a consequence of their normal metabolic activity, a wide variety of antibiotic metabolites and volatile compounds that can negatively affect pathogen metabolism, and furthermore, it is possible to manipulate the extent of biocontrol activity exerted in controlled environments by manipulation of environmental conditions (e.g., temperature, moisture, pH) and by the supply of nutrients provided. Greenhouse environments are typically precisely controlled to optimize environmental conditions for plant growth and development and by extension, provide optimal environmental conditions for the establishment and maintenance of desired soil and phyllosphere microbial populations associated with said plants. However, although many candidate biocontrol organisms demonstrate excellent disease control under controlled greenhouse conditions, their performance in the field is variable, inconsistent and unpredictable. It is likely that various types of environmental stresses such as drought, temperature, nutrient, light intensity and salinity among others, individually and in combination, negatively affect the expression and functionality of multiple enzymes associated with the biochemical pathways responsible for the synthesis of the various biocontrol substances.

Pseudomonas chlororaphis strain PA23 is a known biocontrol agent able to protect canola from stem rot disease caused by the fungus Sclerotinia sclerotiorum (Lib.) de Bary. A number of metabolites produced by P. chlororaphis strain PA23 are thought to contribute its biocontrol properties, including phenazine 1-carboxylic acid, 2-hydroxyphenazine, pyrrolnitrin as well as several volatile compounds (Fernando et al., 2005, Soil Biology and Biochemistry 37:955-964; Zhang et al., 2006. Canadian Journal of Microbiology 52:476-481). In addition to antibiotics, P. chlororaphis strain PA23 produces protease, lipase, and siderophores that may adversely affect the metabolism of plant pathogens. It appears that the production of disease-suppressive metabolites by biocontrol organisms, is controlled by a multi-tiered network of genetic regulation of the various interrelated biochemical pathways. Prior art documents summarizing studies of various biocontrol Pseudomonas spp. suggest that the key genes involved in regulation of the disease-suppressive metabolites include the GacS/GacA two-component signal transduction system (Chancey et al, 1999; Applied and Environmental Microbiology 68:3308-3314), quorum-sensing (QS) circuits (Chin-A-Woeng et al., 2001, Molecular Plant-Microbe Interactions 14:969-979), and the stationary phase sigma factor RpoS (Girard et al., 2006, Microbiology 152:43-58). PsrA (Pseudomonas sigma regulator) controls rpoS transcription and therefore, has an indirect affect on metabolite production (Girard et al., 2006). A negative regulator of phenazine production, known as RpeA, has been identified in Pseudomonas aureofaciens strain 30-84 (Whistler et al., 2003, Journal of Bacteriology 185:3718-3725). Kahn et al., (2005, Journal of Bacteriology 187: 6517-6527) determined that the phz operon of Pseudomonas fluorescens 2-79, which produces phenazine-1-carboxylate, is preceded by two genes, phzR and phzI, that are homologs of quorum-sensing gene pairs of the luxR-luxI family, and furthermore, that deleting phzR and phzI from strain 2-79 led to the loss of antibiotic production. They concluded that PhzR, with its quormone, activates expression of phzA and phzR and that this activation requires an intact phz box sequence located in the divergent promoter region. Girard et al., (2006) found that constitutive expression of phzR is able to restore phenazine-1-carboxamide and acyl homoserine lactone production in a gacS mutant of P. chlororaphis strain PCL1391, and concluded that the presence of a functional QS system alone is sufficient for expression of the phz operon.

### SUMMARY OF THE INVENTION

The exemplary embodiments of the present invention, at least in preferred forms, are directed to a Pseudomonas chlororaphis LysR-type putative transcriptional regulator gene named “ptrA”, said ptrA gene encoding a regulatory protein (PtrA) useful for regulating production of pyrrolnitrin, methods for manipulating said ptrA gene in vitro and in situ, and compositions comprising said ptrA gene.

According to a preferred embodiment of the present invention, there is provided a bacterial PtrA protein configured to regulate a host's metabolism to provide over-expression and production of disease-suppressive metabolites by the host. In a preferred form, the disease-suppressive metabolites comprise pyrrolnitrin.

According to one aspect, the PtrA protein is configured to regulate at least one other set of genes that affect the host's synthesis of pyrrolnitrin. In a preferred form, said PtrA protein regulates a plurality of genes including at least psrA, rpoS, and phzR genes. Regulation by the PtrA protein preferably occurs at the gene transcription level.

According to another aspect, regulation of the ptrA gene of the present invention is subject to positive autoregulation. In a preferred form, the expression of the ptrA gene is interactive and cooperative with the Gac two-component regulatory system.

According to another preferred embodiment of the present invention, there is provided a method for inserting said ptrA gene into a host's genome for thereby affecting the host's metabolic systems. The host is preferably selected from the group comprising microorganisms and plants. It is preferred that the host is a microorganism. In a preferred form, the host is a bacterium. Alternatively, the host may be a plant and most preferably, a canola plant.

According to yet another preferred embodiment of the present invention, there is provided a method for manipulating the expression of said ptrA gene in a host's metabolic systems. In a preferred form, the method provides means for controllably manipulating said ptrA gene to provide controllable over-expression and production of pyrrolnitrin. In another preferred form, said method provides means for controllably manipulating said ptrA gene to provide controllable over-expression and production of pyrrolnitrin over a wide range of environmental conditions including temperature stresses, moisture stresses, nutritional stresses, pH stresses and salinity stresses.

According to yet another preferred embodiment, there is provided a composition comprising a host organism having a genome containing therein the ptrA gene of the present invention.

According to one aspect, the host organism is a microorganism. In a preferred form, the host is a bacterium. The host bacterial genome may contain therein an indigenous ptrA gene, said ptrA gene controllably by a first method of the present invention. Alternatively, the host bacterial genome may contain therein a ptrA gene inserted by a second method of the present invention, said ptrA gene controllable by the first method of the present invention.

According to another aspect, the host organism is a plant having a genome containing a ptrA gene inserted therein by a third method of the present invention, said ptrA gene controllable by a fourth method of the present invention. In a preferred form, the host is a canola plant.

According to yet another preferred embodiment, there is provided a composition for application to a plant, said composition comprising a Pseudomonas chlororaphis strain containing therein said ptrA gene of the present invention. In a preferred form, the composition is configured for aerial application to a plant. Alternatively, the composition can be configured for application as a pre-plant seed treatment. The pre-plant seed treatment can be configured as a powder or liquid composition for on-seed delivery, or alternatively, as a granular composition for in-furrow delivery during planting.

According to a further embodiment, there is provided a composition comprising a plant seed provided with a seed coat comprising a microorganism having a genome containing therein the ptrA gene of the present invention.

### BRIEF DESCRIPTION OF THE DRAWINGS

The present invention will be described in conjunction with reference to the following drawings, in which:

FIG. 1 is a graph showing the expression of prnA and phzA genes by in Pseudomonas chlororaphis strain PA23 (wild type) and strain PA23-443 (ptrA);

FIG. 2 is a graph showing expression of rpoS (encoding the stationary phase sigma factor) and phzR (a quorum-sensing regulator of phenazine production) in Pseudomonas chlororaphis strain PA23 (wild type) and strain PA23-443 (ptrA);

FIG. 3 is a graph showing the time course of ptrA expression in Pseudomonas chlororaphis strain PA23 (wild type);

FIG. 4 is a graph showing expression of the ptrA gene in Pseudomonas chlororaphis strain PA23 (wild type), strain PA23-443 (ptrA), and strain PA23-314 (gacS);

FIG. 5 is a graph showing the rates of biofilm formation by Pseudomonas chlororaphis strain PA23 (wild type; pUCP23), strain PA23-443 (ptrA⁻; pUCP23) and strain PA23-443 harboring ptrA, gacS, psrA, rpoS and phzR in trans;

FIG. 6 is a graph comparing the effects of Pseudomonas chlororaphis strain PA23 (wild type, pUCP22), ptrA-mutant strain PA23-443 (pUCP22), and ptrA-complemented strain PA23-443 (pUCP22-ptrA) in managing Sclerotinia sclerotiorum ascospore infection of canola plants. Panel A shows the % incidence of leaf infection; Panel B shows the severity of stem rot disease. Column means labeled with the same letter do not differ significantly as determined with Duncan's Multiple Range Test (DMRT; P>0.05);

FIG. 7 is a graph showing the effects of the PA23 wild type, and PA23 wild-type strain harboring additional genes in trans on severity of stem-rot disease in canola plants infected with Sclerotinia sclerotiorum ascospores: (A) disease-infected control plant, (B) disease-infected plant that received treatments with the wild type Pseudomonas chlororaphis strain PA23, (C) disease-infected plant that received treatments with Pseudomonas chlororaphis PA23 provided with prnABCD-pUCP23 (pyrrolnitrin biosynthetic cluster), (D) disease-infected plant that received treatments with Pseudomonas chlororaphis PA23 with inserted ptrA-pUCP23 (transcriptional regulator on high copy number plasmid), (E) disease-infected plant that received treatments with Pseudomonas chlororaphis PA23 with inserted ptrA-pRK415 (transcriptional regulator on low copy number plasmid), (F) disease-infected plant that received treatments with Pseudomonas chlororaphis PA23 with inserted ptrA-pRK415 and prnABCD-pUCP23, and (G) un-infected control plant.

FIG. 8 is a photograph showing the effects of the PA23 derivatives on canola plants infected with Sclerotinia sclerotiorum ascospores: (A) disease-infected control plant, (B) un-infected control plant, (C) disease-infected plant that received treatments with the wild type Pseudomonas chlororaphis strain PA23, and (D) disease-infected plant that received treatments with Pseudomonas chlororaphis PA23 provided with prnABCD-pUCP23 (pyrrolnitrin biosynthetic cluster);

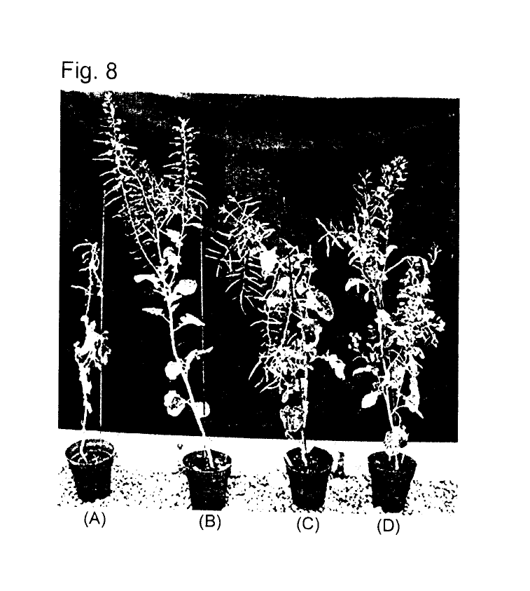

FIG. 9 is a photograph showing the effects of the PA23 derivatives on canola plants infected with Sclerotinia sclerotiorum ascospores: (A) disease-infected control plant, (B) disease-infected plant that received treatments with the wild type Pseudomonas chlororaphis strain PA23, (C) disease-infected plant that received treatments with Pseudomonas chlororaphis PA23 provided with prnABCD-pUCP23 (pyrrolnitrin biosynthetic cluster), (D) disease-infected plant that received treatments with Pseudomonas chlororaphis PA23 with inserted ptrA-pRK415 (transcriptional regulator on low copy number plasmid), (E) disease-infected plant that received treatments with Pseudomonas chlororaphis PA23 with inserted ptrA-pUCP23 (transcriptional regulator on high copy number plasmid), and (F)) disease-infected plant that received treatments with Pseudomonas chlororaphis PA23 with inserted ptrA-pRK415 and prnABCD-pUCP23; and

FIG. 10 is a flow chart illustrating PtrA-affected regulation of metabolic pathways in Pseudomonas chlororaphis strain PA23. Abbreviations are: PRN, pyrrolnitrin; PCA, phenazine-1-carboxylic acid; 2-OH-PHZ, 2-hydroxyphenazine.

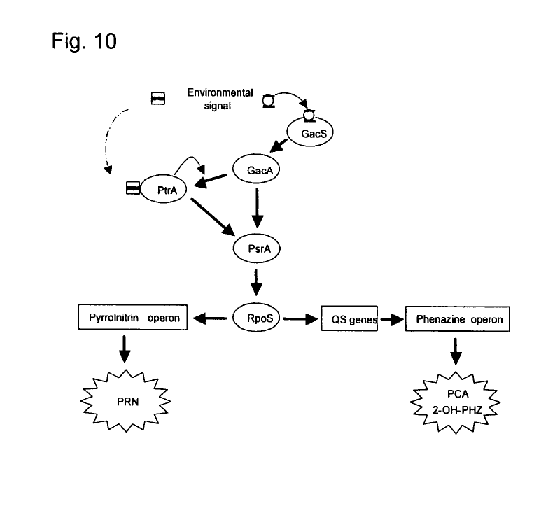

### DETAILED DESCRIPTION OF THE INVENTION

The present invention provides a Pseudomonas chlororaphis LysR-type putative transcriptional regulator gene set forth in SEQ ID NO: 1 and named “ptrA” for Pseudomonas transcriptional regulator [Genbank accession number AAY90576]. Expression of the ptrA gene produces a protein comprising the amino acid sequence set for in SEQ ID NO: 2, and named PrtA. We have surprisingly discovered the PrtA protein controls regulation of microbial production of the antibiotic metabolite pyrrolnitrin, and that the prtA gene can be manipulated to overexpress microbial production of pyrrolnitrin for positively affecting biocontrol of fungal plant pathogens, and can provide increased metabolic fitness to microorganisms in nutrient deprivation conditions. We have also discovered that the ptrA gene positively affects the formation of biofilms comprising complex aggregations of microorganisms contained within and about protective extracellular matrices of polymeric substances. The ptrA gene according to the present invention is subject to positive autoregulation and is also dependent upon the Gac two-component regulatory system. The ptrA gene according to the present invention regulates several genes associated with the expression of genes associated with the production of antibiotic metabolites, including psrA, rpoS, and phzR.

The discovery, identification and characterization of the ptrA gene, its effects on microbial antibiotic production, its relationship with other important regulatory elements (e.g., GacS, PsrA, RpoS, QS), and its use for suppression of plant pathogens are described in more detail in the following examples.

### Examples

### Bacterial Strains, Plasmids, Media and Culture Conditions

The bacterial strains used in these examples are listed in Table 1.

TABLE 1
Bacterial strains.

| | | Source or |
|---|---|---|
| Strain | Relevant genotype or phenotype | reference |
| Pseudomonas | | |
| chlororaphis | | |
| PA23 | Phz⁺Rif^{R} wild type (soybean plant isolate) | Reference 1 |
| PA23-443 | Phz⁻Rif^{R} ptrA::Tn5-OT182 genomic | This study |
| | fusion | |
| PA23-443 | Phz⁻ Rif^{R} ptrA::Tn5-OT182 genomic | This study |
| (pUCP22-ptrA) | fusion, ptrA-complemented strain | |
| PA23-443 | Phz⁻ Rif^{R} ptrA::Tn5-OT182 genomic | This study |
| (pUCP23-gacS) | fusion, containing gacS in trans | |
| Escherichia | | |
| coli | | |
| DH5α | supE44 ΔlacU169 (φ80 lacZΔM15) | Reference 2 |
| | hsdR17 recA1 endA1 gyrA96 thi-1 relA1 | |
| SM10 | Mobilizing strain; RP4 tra genes | Reference 3 |
| | integrated in chromosome; Km^{R}Tc^{R} | |

Reference 1: Savchuk et al., 2004, FEMS Microbiology Ecology 49: 379-388.
Reference 2: GIBCO ® (registered trade mark of Invitrogen Corp.), Invitrogen Canada Inc., 2270 Industrial St., Burlington, ON, Canada
Reference 3: Simon et al., 1983. Bio/Technology 1: 784-791.

The plasmids used in these examples are listed in Table 2.

TABLE 2
Plasmids.

| | | Source or |
|---|---|---|
| Plasmid | Relevant genotype or phenotype | reference |
| pOTI82 | pSUP102(GM)::Tn5-OT182 Cm^{R} Gm^{R} Amp^{R} Tc^{R} | Reference 4 |
| pOT182-443 (XhoI) | pOT182 containing gacS::Tn5-OT182 genomic | This study |
| | fusion | |
| pCR^{R}2.1TOPO | Cloning vector for PCR products | Reference 2 |
| pUCP22 | Broad-host-range vector; IncP OriT, Amp^{R} Gm^{R} | Reference 5 |
| pCR-phzAR | pCR^{R}2.1TOPO containing the entire phzR gene and | This study |
| | the 5′ end of phzA from P. chlororaphis PA23 | |
| pUCP22-phzR | pUCP22 containing phzR from P. chlororaphis | This study |
| | PA23 | |
| pCR-psrA | pCR^{R}2.1TOPO containing psrA from P. chlororaphis | |
| | PA23 | |
| pUCP22-ptrA | pUCP22 containing ptrA from P. chlororaphis | This study |
| | PA23 | |
| pPTRA-lacZ | pLP170 containing the ptrA promoter region from | This study |
| | P. chlororaphis PA23 | |
| pPHZA-lacZ | pLP170 containing the phzA promoter region from | This study |
| | P. chlororaphis PA23 | |
| pPHZR-lacZ | pLP170 containing the phzR promoter region from | This study |
| | P. chlororaphis PA23 | |
| pPSRA-lacZ | pLP170 containing the psrA promoter region from | This study |
| | P. chlororaphis PA23 | |
| pLP170 | Promoterless lacZ transcriptional fusion vector | Reference 6 |
| pME3219 | pME6010 containing an hcnA-lacZ translational | Reference 7 |
| | fusion | |

Reference 4: Merriman et al., 1993, Gene 126: 17-23.
Reference 5: West et al., 1994, Gene 148: 81-86.
Reference 6: Preston et al., 1997, Infection and Immunity 65: 3086-3090.
Reference 7: Laville et al., 1998. Journal of Bacteriology 180: 3187-96.

The primers used in these examples are listed in Table 3.

TABLE 3

Primers

| | | Source or | |
|---|---|---|---|
| Primer | Sequence listing | reference | SEQ ID NO |
| ptrA-F | 5′-gggaaccggcttatagcca-3′ | This study | SEQ ID NO: 3 |
| ptrA-R | 5′-atccagttgctggagcgtatt-3′ | This study | SEQ ID NO: 4 |
| ptrA-F2 | 5′-aagtacggggcgtaactgtc-3′ | This study | SEQ ID NO: 5 |
| ptrA-R2 | 5′-cggcctttttcagcaggtt-3′ | This study | SEQ ID NO: 6 |
| phzAR-F | 5′-aatcctgccatccaactc-3′ | This study | SEQ ID NO: 7 |
| phzAR-R | 5′-aagttgttcgaaggggttca-3′ | This study | SEQ ID NO: 8 |
| psrA-F | 5′-cttggcaatcctcctttttc-3′ | This study | SEQ ID NO: 9 |
| psrA-R | 5′-tagcttagcggatgtaagctg-3′ | This study | SEQ ID NO: 10 |
| psrA- | 5′-ccggatccggtgacgccggtttca-3′ | This study | SEQ ID NO: 11 |
| BamH1 | | | |
| RpoS-R | 5′-cagcagggttttatccgaat-3′ | This study | SEQ ID NO: 12 |
| short | | | |
| TNP5- | 5′-accatttcaacggggtctcac-3′ | Reference 9 | SEQ ID NO: 13 |
| FORWARD | | | |
| TNP5- | 5′-tgactccatgtgacctccta-3′ | Reference 9 | SEQ ID NO: 14 |
| REVERSE | | | |
| Tn5-0N82 | 5′-gatcctggaaaacgggaaagg-3′ | Reference 9 | SEQ ID NO: 15 |
| Tn5-OT182 | 5′-atgttaggaggtcacatg-3′ | Reference 9 | SEQ ID NO: 16 |
| right | | | |

Reference 9: Poritsanos et al., 2006,

### Example 1

### Generation, Screening and Characterization of P. chlororaphis Strain PA23 Mutants

Escherichia coli strains were cultured at 37° C. on Lennox Luria Bertani (LB) agar (BD Biosciences, 2280 Argentia Road, Mississauga, ON. Canada). Pseudomonas chlororaphis PA23 and its derivatives were cultured at 28° C. on LB agar, M9 minimal media (BD Biosciences), Minimal M9 casamino acid (M9CA; BD Biosciences), Terrific broth (Sambrook et al., 1989, Molecular Cloning: A Laboratory Manual, 2nd Ed., Cold Spring Harbor Laboratory, Cold Spring Harbor, N.Y) or Peptone Tryptic soy broth (PTSB; Ohman et al., 1980, Journal of Bacteriology 142:836-842). As required, media were supplemented with the following antibiotics: tetracycline (Tc; 15 μg/mL), gentamicin (Gm; 15 μg/mL), ampicillin (Amp; 100 μg/mL) for E. coli, and rifampicin (Rif; 25 μg/mL), Tc (15 or 100 μg/mL), Gm (25 μg/mL), piperacillin (13 or 500 μg/mL) for P. chlororaphis. All antibiotics were obtained from Research Products International Corp. (Mt. Prospect, Ill.).

PCR. Polymerase Chain Reaction (PCR) was performed under standard conditions as suggested by Invitrogen Life Technologies data sheets supplied with their Taq polymerase.

Nucleic acid manipulation. Cloning, purification, electrophoresis, and other manipulations of nucleic acid fragments and constructs were performed using standard techniques as provided in Sambrook et al., (1989). To clone the PA23 ptrA gene, oligonucleotide primers ptrA-F and ptrA-R (Table 1) were used to amplify a 2.2-kb product which was cloned into vector pCR2.1-TOPO following manufacturer's instructions. The 2.2-kb ptrA insert was then excised with XbaI and BamHI and cloned into the same sites of pUCP22, generating pUCP22-ptrA. To generate a ptrA-lacZ transcriptional fusion, the ptrA promoter region was PCR amplified using primers ptrA-F2 and ptrA-R2 (Table 1). The 1.3-kb PCR product was cloned into pCR2.1-TOPO. The ptrA promoter was then excised using XhoI-HindIII and subcloned into the same sites of pLP170, generating pPTRA-lacZ. phzA and phzR are divergently transcribed and the promoter regions of both genes lie within a 428-bp intergenic region. Using primers phzAR-F and phzAR-R (Table 1), a 1.68-kb fragment containing the entire phzR gene and the 5′ end of phzA was PCR amplified and cloned into pCR2.1-TOPO, creating pCR-phzAR. pCR-phzAR was digested with HindIII and XbaI and the 1.68-kb insert was cloned into the same sites of pUCP22, creating pUCP22-phzR. A 1.1-kb HindIII and EcoRV fragment containing the phzA promoter was excised from pCR-phzAR and cloned into HindIII-SmaI digested pLP170, forming pPHZA-lacZ. Plasmid pPHZR-lacZ was constructed by EcoRI-EcoRV digestion of pCR-phzAR and subcloning the 1.1-kb fragment into EcoRI-SmaI cut pLP170. The PA23 psrA gene was amplified using primers psrA-F and psrA-R and the 950 by PCR fragment was cloned into pCR2.1-TOPO, creating pCR-psrA. The insert was removed as a HindIII-EcoRV fragment and ligated into the HindIII-SmaI sites of pUCP22 (pUCP22-psrA). To generate a psrA-lacZ transcriptional fusion, primer psrA-BamH1 (Table 1) and the M13 forward universal primer were used to PCR amplify a 960-bp product from pCR-psrA. The DNA was digested with EcoR1 and BamH1 and cloned into the same sites of pLP170 creating pPSRA-lacZ.

Tn5-OT182 transposon mutagenesis. Bacterial conjugations were performed to introduce Tn5-0T182 into P. chlororaphis PA23 by biparental mating following the method of Lewenza et al., (1999, Journal of Bacteriology 181:748-756). For each mating, 5-10 Tc^{R} colonies were screened by PCR to ensure that transconjugants contained a Tn5 insertion using TNP5-FORWARD and TNP5-REVERSE primers (Table 1). To determine the site of Tn5-OT182 insertion rescue cloning was performed following previously described methods (Lewenza et al., 1999).

Sequence analysis. Plasmids isolated from Tc^{R} XhoI clones were sent for sequencing using oligonucleotide primer Tn5-ON82 (Table 1), which anneals to the 5′ end of Tn5-OT182. BamHI or ClaI rescue plasmids were sequenced using primer Tn5-OT182 right (Table 1), which anneals to the 3′ end of the transposon. All sequencing was performed at the University of Calgary Core DNA Services facility (Faculty of Medicine, University of Calgary, 3350 Hospital Drive NR, Calgary, AB Canada). Sequences were analyzed using blastn and blastx databases.

Nucleotide sequence accession number. The GenBank accession number for the P. chlororaphis PA23 ptrA gene sequence is EF054873.

Isolation of a P. chlororaphis mutant deficient in antifungal activity. Approximately 4000 transconjugants were screened in radial diffusion plate assays to identify mutants exhibiting increased or decreased antifungal activity compared to the wild type. One mutant was identified, PA23-443, that exhibited no antifungal activity (Table 4). Southern blot analysis revealed a single Tn insertion in PA23-443 (data not shown).

TABLE 4
Phenotypic characterization of Pseudomonas chlororaphis PA23, Tn5 mutant
PA23-443, strain PA23-443 containing ptrA, gacS, rpoS, phzR and psrA in
trans and strain PA23-314 (gacS) containing ptrA in trans.

| | | | Extracellular Metabolite | | | |
|---|---|---|---|---|---|---|
| Strain (gene | | | Activity Protease^{b} | | | |
| provided in trans) | Color^{a} | Antifungal^{b} | 24 h | 48 h | Lipase^{c} | HCN |
| PA23 (vector) | orange | 7.8 (1.6) | 12.0 (0.5) | 16.5 (0.5) | + | + |
| PA23-443 (vector) | white | 0.0 | 0.0 | 9.2 (1.0) | + | − |
| PA23-443 (ptrA) | orange | 8.6 (1.0) | 12.5 (0.3) | 17.0 (0.5) | + | + |
| PA23-443 (gacS) | orange | 6.8 (0.8) | 11.8 (0.8) | 19.0 (1.3) | + | + |
| PA23-443 (psrA) | white | 0.0 | 0.0 | 9.5 (0.5) | + | + |
| PA23-443 (rpoS) | white | 0.0 | 0.0 | 7.8 (1.3) | + | + |
| PA23-443 (phzR) | white | 0.0 | 0.0 | 8.8 (1.6) | + | + |
| PA23-314 (vector) | white | 0.0 | 0.0 | 0.0 | − | − |
| PA23-314 (ptrA) | white | 0.0 | 0.0 | 0.0 | − | − |
| PA23-314 (psrA) | white | 0.0 | 0.0 | 0.0 | − | − |

^{a}Color of bacterial colonies on PDA plates.
^{b}Mean (standard deviation) of the zones of activity (mm) obtained from six replicates.
^{c}Lipase activity at 24 h.

Sequence analysis of DNA flanking the Tn insertion in mutant PA23-443 showed 89% identity at the amino acid level to a Pseudomonas fluorescens LysR-type putative transcriptional regulator [Genbank accession #AAY90576]. This newly identified gene was named ptrA for Pseudomonas transcriptional regulator. The PA23 ptrA gene was amplified using PCR and cloned into pUCP22 for PA23-443 complementation. The presence of ptrA in trans restored antifungal activity to that of the wild type (Table 4) confirming that the PA23-443 phenotype results from ptrA inactivation.

### Example 2

### Effects of PtrA Gene Regulation on the Production of Antibiotic Metabolites by P. Chlororaphis Strain Pa23 Mutants

Antifungal assays. Radial diffusion assays to assess fungal inhibition in vitro. aliquots of overnight bacterial cultures were spotted onto PDA plates (Difco®), 0.5 cm was from the edge of the plates. The bacteria were allowed to grow for 16 h at 28° C. before a 0.6-cm fungal plug of S. sclerotiorum was placed on the center of the plate. Plates were incubated at room temperature and antifungal activity was assessed after 3-4 days by measuring the distance between the edges of the colony and the fungal mycelium. Four replicates were analyzed for each strain and assays were repeated three times.

PtrA regulates antibiotic production. Wild-type strain PA23 produces the compounds phenazine 1-carboxylic acid and 2-hydroxyphenazine, resulting in an orange phenotype (Zheng et al, 2006, Canadian Journal of Microbiology 52:476-481). When growing on plates strain PA23-443 is white in color and only turns pale orange after at 72 h, suggesting phenazine production is markedly reduced in a ptrA-deficient background. Spectral analysis of culture extracts confirmed this was the case. For the PA23 wild type and complemented mutant, we were able to observe the characteristic 367-nm peak, reflective of total phenazine production (data not shown). In PA23-443, this peak was absent in both 24h- and 72h-culture extracts (data not shown). Our pyrrolnitrin analyses yielded similar findings. Pyrrolnitrin was detected in culture extracts of PA23 (120.3±21 μg/10¹² cfu) and PA23-443 (pUCP22-ptrA) (162.3±30 μg/10¹² cfu); whereas in PA23-443 cultures, this antibiotic was below the detectable limit (<10 μg/10¹² cfu). The increased pyrrolnitrin expression in PA23-443 (pUCP22-ptrA) is likely caused by a gene dosage effect.

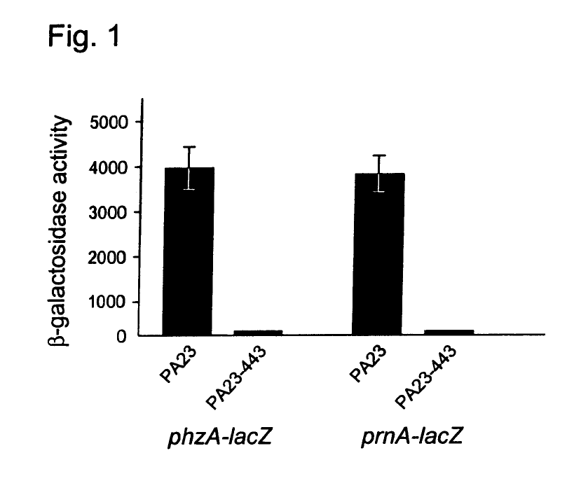

To substantiate findings from our antibiotic analysis, gene expression studies were undertaken. We monitored expression of phzA-lacZ (phenazine) and prnA-lacZ (pyrrolnitrin) in PA23 and the ptrA mutant. As can be seen in FIG. 1, at 24 h, phzA and prnA transcription were significantly reduced in PA23-443 compared to PA23. At 48 and 72 h, there was no increase in expression of either phzA or prnA in PA23-443 (data not shown).

### Example 3

### Effects of PtrA Gene Regulation on the Production of Phenazine, Pyrrolnitrin and HCN by P. chlororaphis Strain PA23 Mutants

Phenazine analysis. Overnight bacterial cultures grown in LB were subjected to phenazine extraction and quantification by UV-visible light spectroscopy following the method of Chancey et al. (1999). Phenazine analysis was performed three times. A phzA-lacZ transcriptional fusion was used to analyze phenazine gene expression. Cultures of PA23 and PA23-443 harboring the phzA-lacZ fusion were grown for 16 hours in PTSB at which point β-galactosidase activity was determined according to Miller (1972, Experiments in Molecular Genetics, pp. 352-355, Cold Spring Harbor Laboratory, Cold Spring Harbor, N.Y.).

Pyrrolnitrin analysis. The amount of pyrrolnitrin produced by PA23 (pUCP22), PA23-443 (pUCP22) and PA23-443 (pUCP22-ptrA) was quantified by high-pressure liquid chromatography (HPLC). Each strain was grown in 10 ml of 523 media on the rotary shaker for 4 days. Cell density for each strain was adjusted to 10⁹ cfu/ml and 10 ml of the adjusted volume was extracted with ethyl acetate according to the method of Hwang et al. (2002, Biological Control 25: 56-63). Samples were re-dissolved in 300 μL of methanol and 90 μL aliquots of each sample were injected into a Gemini C₁₈ column (100×4.6 mm; 5-μm particle diameter) (Phenomenex Inc, 411 Madrid Avenue, Torrance, Calif.) and analyzed in a gradient of 90% eluent A (water) and 10% eluent B (acetonitrile) at 0 min, increasing eluent B to 100% after 18 min. The eluent flow rate was 1.0 ml per min. Peaks were detected by UV absorption at 254 nm using a Waters Multi Fluorescence Detector (Waters Corporation, Milford, Mass.). The concentration of pyrrolnitrin in each sample was based on standard curves prepared from purified pyrrolnitrin (Sigma). HPLC-grade solvents were obtained from Fisher Scientific. A prnA-lacZ transcription fusion was used to monitor pyrrolnitrin gene expression in PA23 and PA23-443. Cultures were grown in PTSB for 16 hours (OD_{600 nm}=2.5-3.0), at which point prnA expression was assessed using β-galactosidase assays (Miller, 1972) Cultures were analyzed in triplicate and the experiments were repeated three times.

HCN analysis. Production of hydrogen cyanide was determined qualitatively using Cyantesmo paper (Machery-Nagel GmbH & Co., Düren, Germany). To monitor expression of the genes encoding hydrogen cyanide, plasmid pME3219 containing an hcnA-lacZ translational fusion was transformed into PA23 and PA23-443. Cultures were grown in PTSB until they reached stationary phase (OD_{600 nm}=2.5-3.0), at which point hcnA expression was assessed using β-galactosidase assays (Miller, 1972). Samples were analyzed in triplicate and experiments were repeated three times.

### Effects of PtrA on Metabolite Production

In addition to the diffusible antibiotics phenazine and pyrronitrin, PA23 produces a number of metabolites that likely contribute to biocontrol, including HCN, protease, and lipase molecules (Poritsanos et al., 2006). Using cyanotesmo paper, we were able to detect HCN production by PA23 and PA23-443 (pUCP22-ptrA), but not PA23-443 (data not shown). These findings were supported by hcnA-lacZ expression studies. Expression of an hcnA-lacZ translational fusion was negligible in PA23-443 (110±8) compared to the PA23 wild type (18,227±2,796). Production of other extracellular metabolites is summarized in (Table 4). At 24h, protease activity could be detected for the wild type; however for PA23-443, protease activity was not observed until 48 h. Therefore, production of protease appears to be delayed in a ptrA-deficient background. Addition of ptrA in trans restored the wild-type phenotype. Lipase activity, which is observed as a white precipitate around the bacterial colony on lipase plates, cannot be detected until after 48 h. We did not see any difference in lipase production between PA23, PA23-443 and PA23-443 (pUCP22-ptrA) (Table 4).

### Example 4

### Effects of PtrA Gene Regulation on the Expression of PsrA, RpoS and PhzR by P. chlororaphis Strain PA23 Mutants

RpoS Expression. An rpoS-lacZ transcriptional fusion was used to monitor expression of this gene in PA23 and PA23-443. β-galactosidase assays were performed in triplicate after cultures had reached an OD₆₀₀ of 1.0. RpoS protein levels were determined by Western blot analysis using RpoS-specific antisera following previously described methods (Poritsanos et al., 2006). RpoS was quantified with a Fluorochem 2000 Phosphoimager using Fluorochem Stand Alone software, Version 2.0.

Exoproduct analyses. Production of homoserine lactone (HSL) autoinducer molecules was assessed qualitatively by spotting 5 μL of an overnight culture onto Chromobacterium violaceam CV026-seeded plates. CV026 is an autoinducer-deficient strain that turns purple in the presence of exogenous C₄-, C₆-, C₈-HSL due to the production of the quorum-sensing controlled pigment violacein. Extracellular protease activity was determined by inoculating 5 μL of an overnight culture onto 2% skim milk-agar plates. Proteolysis was observed as zones of lysis around the colony after 24-36 h at 28° C. Lipase activity was detected using the protocol of Lonon et al., (1988, Journal of Clinical Microbiology 26: 979-984). Lipase activity was indicated by a zone of fatty acid precipitation around the colony after 24-72 h. Siderophore production was assayed by spotting a 5-μL aliquot of overnight culture onto Chrome azurol S (CAS) agar plates (Schwyn et al., 1987 Analytical Biochemistry 160:47-56) followed by inculbation for 16 h at 28° C. Data represents the average of six replicates and assays were repeated three times.

Competition experiments. Competition assays were carried out in M9 minimal media (0.2% glucose; 1 mM MgSO₄) to minimize spontaneous gacS accumulation. Overnight cultures of PA23 and PA23-314 were used to inoculate 20 mL of media such that competing strains were present in equal numbers (10⁸ cfu/mL). The mixed culture was grown at 28° C. with shaking for a period of 8 days. Colony-forming-units (cfu) of the wild type and gacS populations were monitored daily by plating serial dilutions onto LB agar plates with and without antibiotics [PA23-314 is Tc^{R} due to the Tn5-OT182 insertion]. Cultures were analyzed in triplicate and the experiment was repeated twice.

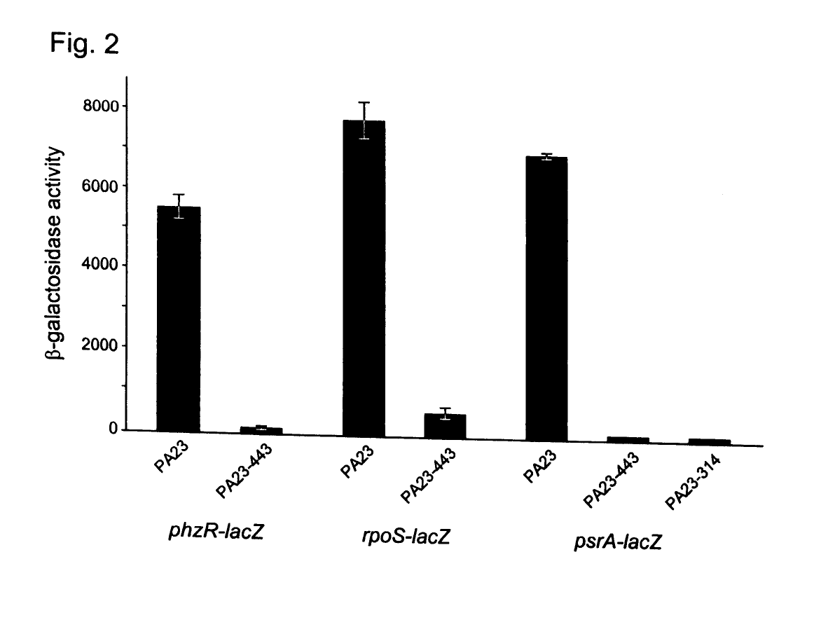

RpoS and PhzR are subject to PtrA regulation. We postulated that PtrA might control expression of other regulatory genes, such as rpoS and phzR, and therefore regulate antifungal metabolite production indirectly. We examined PtrA regulation of the stationary phase sigma factor, RpoS and the QS transcriptional activator PhzR through rpoS-lacZ and phzR-lacZ gene expression analysis. As outlined in FIG. 2, we discovered that PtrA controls expression of both genes.

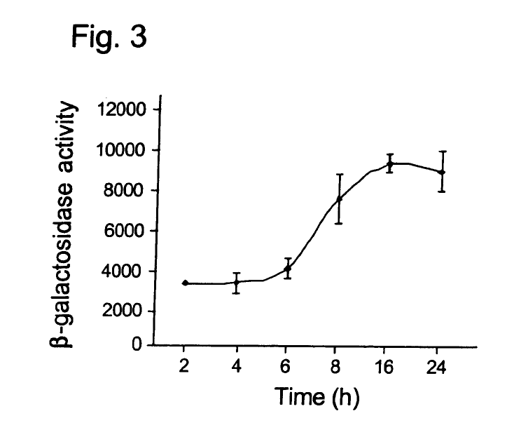

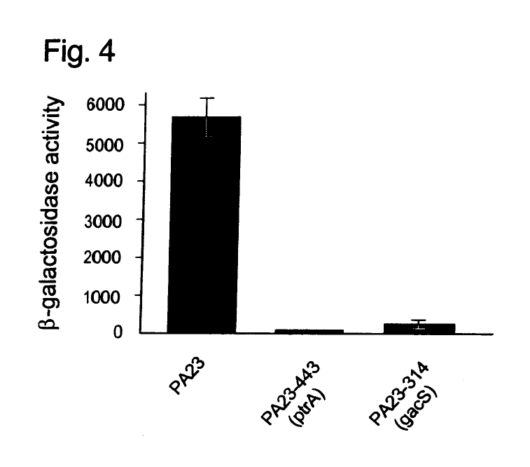

Regulation of PtrA. To better understand factors governing ptrA expression, a ptrA-lacZ transcriptional fusion was generated. We determined that ptrA transcription peaks at 16 h (FIG. 3), approximately the point at which cells are entering into stationary phase. As can be seen in FIG. 4, transcription of this fusion was negligible in the ptrA-mutant background, indicating ptrA is subject to positive autoregulation. As ptrA expression is extremely low in the gacS-mutant PA23-314, we conclude that this gene is also under control of the Gac two-component regulatory system.

GacS is able to complement the ptrA mutation. We investigated whether providing other regulatory genes in trans would complement the ptrA mutation. A number of genes were examined, including gacS, ptrA, rpoS and phzR, all of which were cloned under control of the constitutive lac promoter on plasmid pUCP22 or pUCP23. The only gene able to complement PA23-443 was gacS. Addition of pUCP23-gacS resulted in a white to orange color change (data not shown), and restored antifungal, protease, and autoinducer activity to that of wild type (Table 4). However when the reverse experiment was attempted, i.e. expressing ptrA in trans in a PA23 gacS mutant [PA23-314 (pUCP22-ptrA)], ptrA was unable to complement the gacS mutation (data not shown).

### Example 5

### Effects of PtrA Gene Regulation on Motility and Biofilm Production by P. chlororaphis Strain PA23 Mutants

Motility analysis. Flagellar (swimming) and swarming motility were monitored by inoculating 5 μL of an overnight culture onto either LB or M9CA-media solidified with 0.3% agar. After 20 h and 36 h incubation at 28° C., the diameter of the swim zone was measured. Swarming motility was assayed by inoculating bacterial cells with an applicator stick onto the surface of a Swarm (WM) media plate (0.5% peptone, 0.3% yeast extract, 0.5% agar), previously air-dried for 2 h. Results were obtained after 16-30 h incubation at 28° C. Twitching mobility was assessed on LB and M9CA plates containing 1% agar. Bacterial cultures were stabbed to the bottom of the plates and then incubated for 72 h after which, twitch zones were measured. For motility assays, five replicates were analyzed and the experiment repeated three times.

Biofilm development. We employed a highly-reproducible 96-well plate assay (O′Toole and Kolter, 1998, Molecular Microbiology 28:449-461) to assess the ability of PA23 (pUCP22), PA23-443 (pUCP22), PA23-443 (pUCP22-ptrA), PA23-443 (pUCP22-gacS), PA23-443 (pUCP22-psrA), PA23-443 (pUCP22-rpoS) and PA23-443 (pUCP22-phzR) to form biofilms.

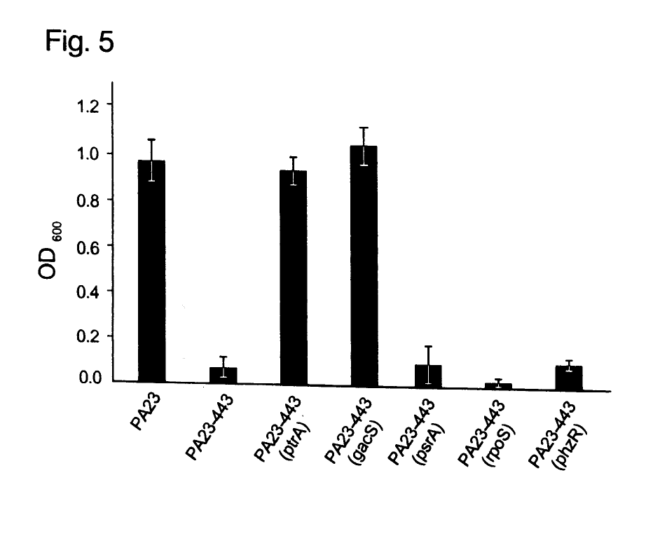

PtrA affects biofilm formation and motility. In a previous study, we discovered that a gacS mutation causes a ten-fold reduction in biofilm formation in PA23, together with a decreased rate of swimming and swarming motility (Poritsanos et al., 2006). Consequently, we examined whether the same would hold true for a PA23 ptrA mutant. As observed in FIG. 5, biofilm formation by PA23-443 (ptrA) is greatly diminished compared to the wild type. Complementation with either ptrA or gacS in trans, restored biofilm development to parental levels (FIG. 5). Conversely, psrA, rpoS, and phzR had no affect on the ability of this mutant to establish a biofilm (FIG. 5).

Next, we investigated whether PA23-443 would demonstrate altered motility which in turn might contribute to its diminished biofilm formation and antifungal activity. Flagellar motility was assessed at 20 and 48 h. As outlined in Table 5, the rate of PA23-443 (pUCP22) swimming motility was slightly decreased compared to PA23 (pUCP22), and the mutant complemented with either ptrA or gacS in trans. We have repeated the flagellar motility analyses numerous times and consistently see the same trend; a modest but reproducible drop in the rate of PA23-443 swimming motility. Addition of psrA, rpoS and phzR in trans does not restore swimming to wild-type levels (Table 5).

Because of the irregular pattern of swarming, we did not perform quantitative analyses. One major difference we were able to observe was that the ptrA mutant was delayed in initiation of swarming motility compared to the wild type and complemented mutant. Swarming initiation is observed as fork-like tendrils extending out from the colony. The second difference we noted was the PA23-443 swarm pattern, which was less irregular that that of the parent, resembling swimming more than swarming motility.

TABLE 5
Flagellar motility analysis of Pseudomonas chlororaphis PA23,
Tn5 mutant PA23-443 and mutant PA23-443 with ptrA, gacS,
psrA, rpoS and phzR in trans.

Swim Zone
Diameter (mm)

| Strain | 24 h^{a} | 48 h^{a} |
|---|---|---|
| PA23 (pUCP23) | 34.8 (0.6) | 70.8 (1.7) |
| PA23-443 (pUCP23) | 30.7 (1.0) | 54.0 (3.2) |
| PA23-443 (pUCP23-ptrA) | 34.5 (1.2) | 65.8 (1.9) |
| PA23-443 (pUCP23-gacS) | 37.1 (2.1) | 70.9 (2.3) |
| PA23-443 (pUCP22-psrA) | 31.1 (1.5) | 55.9 (1.4) |
| PA23-443 (pUCP22-rpoS) | 30.9 (1.1) | 61.6 (1.8) |
| PA23-443 (pUCP22-phzR) | 31.3 (0.8) | 56.5 (0.5) |

^{a}Mean (standard deviation) of swim zones from five replicates.

A ptrA mutation does not confer a GASP phenotype. A Pseudomonas fluorescens isolate has been described (Silby et al., 2005) with a mutation in a LysR-type regulatory gene (finR) that resulted in a loss of AF activity. This mutant displayed a growth advantage in stationary phase (GASP) phenotype, enabling it to overtake the wild type during prolonged batch culture (Silby et al., 2005). To determine if a ptrA deficiency imparts a GASP phenotype, competition experiments were undertaken. A 1:1 mixed culture of PA23 and PA23-443 was established and allowed to grow for 8 days. Daily enumeration of culture viability revealed equivalent numbers of PA23 and PA23-443 survivors (data not shown). Thus it appears that a ptrA-mutation does not impart a fitness advantage over the wild type during long-term batch culture.

### Example 6

### Effects of PtrA Gene Regulation on Biocontrol of Stemrot of Canola P. chlororaphis Strain PA23 Mutants Under Growth Chamber Conditions

Biocontrol under greenhouse conditions. Strains PA23 (pUCP22), PA23-443 (pUCP22) and PA23-443 (pUCP22-ptrA) were assessed for their efficiency in suppressing stem rot of canola [Brassica napus (cv. Westar)] under greenhouse conditions. Brassica napus (cv. Westar) plants were grown in pots (21 cm×20 cm) at 24/16° C. with a 16-h photoperiod. The plants were sprayed at 30% and 50% flowering (double spray) with bacterial strains (2.0×10⁸ cfu/mL) suspended in 100 mM phosphate buffer, pH 7.0 with Tween20, and maintained in a humidity chamber (24/16° C. with a 16-h photoperiod). Twenty four hours after bacterial inoculation, canola petals were sprayed with ascospores of S. sclerotiorum (8×10⁴ spores/mL) suspended in 100 mM phosphate buffer, pH 7.0 containing 0.02% Tween 20. The pathogen control plants were inoculated with ascospores, while the healthy control plants were sprayed with phosphate buffer. All plants were inoculated in a humidity chamber. Fourteen days after inoculation with S. sclerotiorum ascspores, symptom development was observed and recorded using a 0-7 scale (0=no lesions on the stem; 1=leaf lesion with no stem symptom; 2=1-20 cm stem lesion; 3=21-40 cm stem lesion; 4=41-60 cm stem lesion; 5=61-80 cm stem lesion; 6=81-100 cm stem lesion; 7=>100 cm stem lesion or plant death). Based on symptom development, percent leaf incidence by Sclerotinia (PLI) and stem rot disease severity (DS) were calculated. Eight plants were assessed for each treatment. For assessing infection on leaves, the first 10 leaves from top to bottom, were scored for the presence of the symptom per plant.

$$PLI = \frac{Number of leaves infected by Sclerotinia}{Number of leaves observed} \times 100$$

$$DS = \frac{Total points for all plants using the 0 \text{-} 7 scale}{Number of plants observed}$$

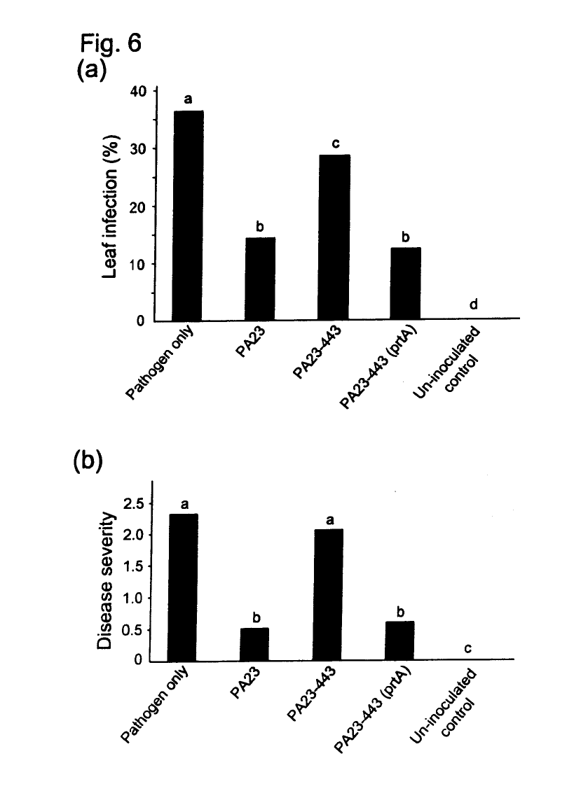

PtrA is essential for biocontrol of S. sclerotiorum in greenhouse studies. The wild-type PA23 (pUCP22), PA23-443 (pUCP22) and PA23-443 (pUCP22-ptrA) were evaluated for their antifungal action against canola stem rot disease caused by S. sclerotiorum. Two parameters were evaluated; 1) incidence of leaf infection and 2) stem rot disease severity. The results of our analyses revealed that the wild-type PA23 afforded significant protection against fungal infection of both stems and leaves and dramatically reduced disease severity (FIG. 6). On the other hand, a ptrA mutation dramatically reduced the ability of this bacterium to suppress disease (FIG. 6). Addition of ptrA in trans restored the biocontrol capacity of the mutant to that of the PA23 parent. Our findings indicate that PtrA is essential for effective biocontrol of S. sclerotiorum infection in canola.

### Example 7

### Effects of PtrA Gene Regulation on Growth and Productivity of Canola under Greenhouse Conditions

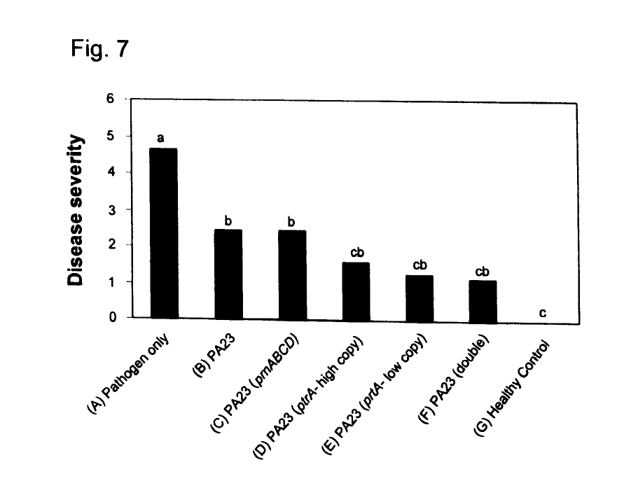

The bacterial strains PA23 (wild type with control vector pUCP23), PA23 (prnABCD-pUCP23; pyrrolnitrin biosynthetic cluster), PA23 (ptrA-pUCP23; transcriptional regulator on high copy number plasmid), PA23 (ptrA-pRK415; transcriptional regulator on low copy number plasmid), and PA23 containing both plasmids (prnABCD-pUCP23)(ptrA-pRK415) were assessed for their efficiency in suppressing stem rot of canola and their effects on canola productivity. Brassica napus (cv. Westar) seeds were sown into pots (21 cm×20 cm) containing a commercial growing mix and then placed into a growth chamber for seed germination and initial plant growth and development. The growth chamber was maintained at 75% humidity, with a 16 h/8 h day/night cycle with temperatures maintained at about 21° during the 16-h day period and 16° C. during the 8-h night period. After the seeds had germinated and the plants reached the 2-leaf stage, they were transferred to the greenhouse (approximately 7 days). When plants had reached 30% flowering (which occurred after approximately 6 weeks of growth) they were moved to a humidity chamber and sprayed with bacterial strains (2.0×10⁸ cfu/ml) suspended in 100 mM phosphate buffer, pH 7.0 with 0.02% Tween 20. The conditions in the humidity chamber were as follows: 16 h photoperiod at 24° C. (day cycle); 8 hours darkness at 16° C. (night cycle); 80% to 90% humidity. Twenty four hours later, plants received a second application of bacteria, as described above. Twenty four hours after bacterial inoculation, canola petals were sprayed with ascospores of S. sclerotiorum (8×10⁴ spores/ml) suspended in 100 mM phosphate buffer, pH 7.0 containing 0.02% Tween 20. The pathogen control plants were inoculated with ascospores, while the healthy control plants were sprayed with phosphate buffer. Two hundred seed pods were then harvested from five plants from each treatment, after which their combined fresh weight was determined. The data in Table 6 show that seed production in canola infected with S. sclerotiorum dropped by about 50% compared to un-infected controls. However, spraying the plants with the wild type PA23 strain or with the PA23 provided with the high copy plasmid, 24 hours prior to infecting the plants with S. sclerotiorum, eliminated the S. sclerotiorum disease effects. Spraying the plants with PA23 provided with (a) the low copy plasmid, (b) both high copy and low copy plasmids, or (c) the pyrrolnitrin biosynthetic cluster 24 hours prior to infecting the plants with S. sclerotiorum, increased seed production relative to the disease-free control plants, by 9%, 13% and 14% respectively (Table 6; FIG. 6; FIG. 7).

TABLE 6
Effects of inoculation with microbial strains provided with the ptrA
gene on seed production by canola exposed to
Sclerotinia infection.

| | Total pod | Fold increase in |
|---|---|---|
| | weight for | pod weight |
| Microbial Inoculation Treatment | 200 pods (g)0 | compared to PA23 |
| Disease-infected control | 30.11 | 0.49 |
| Healthy control (not infected) | 60.28 | 0.98 |
| PA23 wild type | 61.44 | — |
| PA23 provided with prnABCD- | 69.87 | 1.14 |
| pUCP23 (pyrrolnitrin biosynthetic | | |
| cluster) | | |
| PA23 provided with ptrA-pUCP23 | 64.64 | 1.05 |
| (high copy plasmid) | | |
| PA23 provided with ptrA-pRK415 | 67.25 | 1.09 |
| (low copy plasmid) | | |
| PA23 provided with prnABCD- | 69.43 | 1.13 |
| pUCP23 + ptrA-pRK41 (high copy | | |
| & low copy plasmids) | | |

### SUMMARY

Disclosed herein is the characterization of a P. chlororaphis strain PA23 derivative with a mutation in a lysR-type transcriptional regulator, designated PtrA. Unlike the wild type, the ptrA-deficient strain (PA23-443) was unable to protect canola from Sclerotinia stem rot in greenhouse assays, highlighting the importance of PtrA in the biocontrol function of P. chlororaphis strain PA23. Because it would seem that antibiotic production is the primary means by which P. chlororaphis strain PA23 inhibits fungal growth, we examined strain PA23-443 (ptrA) extracts for the presence of phenazines and pyrrolnitrin. We surprisingly discovered that neither antibiotic was produced by this mutant, i.e., strain PA23-443 (ptrA). Expression of prnA-lacZ and phzA-lacZ fusions was negligible in the PA23-443 background indicating regulation occurs at the level of transcription. Next, we investigated whether PtrA exerts control over other regulatory genes, including psrA, rpoS and phzR, and in so doing affects antibiotic production indirectly. A PtrA deficiency significantly reduced expression of all three regulatory genes (FIG. 2), suggesting that PtrA is high up in the regulatory hierarchy of gene expression.

It is known that most LysR-type transcriptional activators are autoregulatory; some members of this family repress their own transcription, while others show positive autoregulation (Schell, 1983, Journal of Bacteriology 153:182-189). PtrA was found to activate its own transcription over sixty-fold (FIG. 4). In addition to being autoregulated, ptrA expression is controlled by the Gac two-component system. A second link to the Gac system was realized with the finding that gacS in trans complements the ptrA mutation (Table 4). It is not clear why multiple copies of gacS restore PA23-443 phenotype to that of the wild type. We anticipate that the increased level of sensor kinase may result in increased phosphotransfer and activation of the response regulator GacA. It is known that GacA regulates gene expression at both the transcriptional and post-transcriptional level (Pessi and Haas, 2001, FEMS Microbiology Letters 200, 73-78). The Gac system is required for expression of psrA in P. chlororaphis PCL1391 (Chin-A-Woeng et al., 2005, Mol. Plant. Microbe Interact. 18:244-253). If both GacA and PtrA transcriptionally activate psrA, the increased level of activated GacA might be able to overcome a PtrA deficiency. This in turn would lead to increased expression of genes downstream of psrA (rpoS, phzR, phzA, etc.). In a recent study, Girard and coworkers (2006) reported that constitutive expression of phzR is able to restore phenazine-1-carboxamide and acyl homoserine lactone production in a gacS mutant. The authors concluded that the presence of a functional QS system alone is sufficient for expression of the phz operon (Girard et al., 2006). In our experiments, constitutive phzR expression did not restore phenazine production in PA23-443, as the strain did not undergo a white to orange color change, nor were we able to detect the presence of phenazines in culture extracts (data not shown). Furthermore, in PA23-443 (pUCP22-p hzR), protease and AF activity were not returned to wild-type levels (Table 4). When we performed the same experiments with our PA23 gacS mutant, PA23-314, our findings were identical.

It is well recognized that in nature the bulk of bacterial biomass does not exist as unicellular organisms living in a planktonic state, but rather as an attached community of cells known as a biofilm (Costerton et al., 1995, Annual Reviews in Microbiology 49:711-745). This adherent cell population is encased in an extracellular matrix that affords protection from environmental agents that would otherwise threaten their planktonic counter parts. Pseudomonas chlororaphis PA23 is able to form thick biofilms on the surface of PVC microtitre plates (FIG. 5). The same trait was observed for PtrA; a ptrA deficiency results in a dramatic reduction in the ability of this bacterium to form biofilms. Complementation with either ptrA or gacS, but not rpoS, psrA or phzR, restored robust biofilm formation (FIG. 5

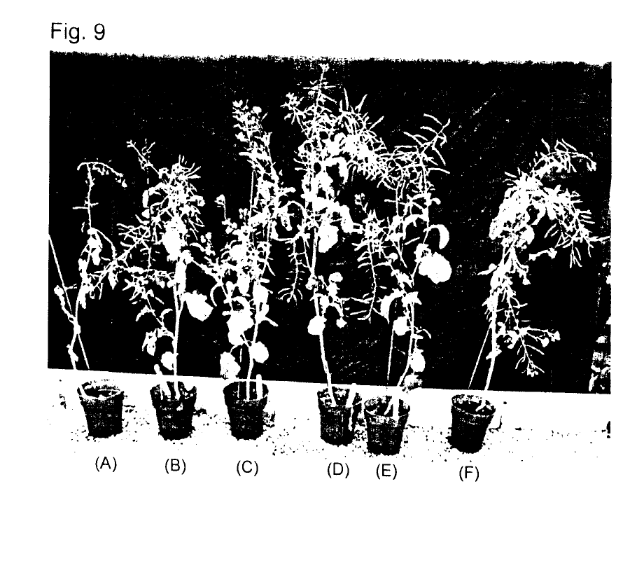

In FIG. 9, we present a model of PtrA regulation based on our discovery of the ptrA gene and its roles in affecting the regulation, expression and production anti-pathogenic metabolites by biocontrol organisms. The GacS/GacA two-component system is positioned at the top of the regulatory hierarchy. GacS is a histidine kinase that responds to an unknown environmental signal, resulting in autophosphorylation and phosphotransfer to the response regulator GacA (Heeb and Haas, 2001, Molecular Plant-Microbe Interactions 14:1351-1363). Once activated, GacA induces expression of downstream genes, including psrA, rpoS and ptrA. In addition to being under control of the Gac system, ptrA is positively autoregulated. Several genes are under PtrA control including the pyrrolnitrin and phenazine biosynthetic operons as well as psrA, rpoS and phzR. In other Pseudomonas spp., RpoS positively controls pyrrolnitrin and phenazine production (Girard et al., 2006; Sarniguet et al., 1995, Proceedings of the National Academy of Science, USA. 92:12,255-12,259). Therefore, we have included RpoS as a positive regulator of PA23 antibiotic production in our model.

We claim:

1. An isolated nucleic acid molecule comprising a nucleotide sequence set forth in SEQ ID NO: 1, said nucleotide sequence encoding a protein inhibitory to Sclerotinia spp.

2. An isolated nucleic acid molecule according to claim 1, wherein said nucleotide sequence encodes a protein comprising an amino acid sequence set forth in SEQ ID NO: 2.

3. A protein inhibitory to Sclerotinia spp., said protein comprising an amino acid sequence set forth in SEQ ID NO: 2.

4. A nucleotide construct comprising a nucleic acid molecule of claim 1, wherein said nucleic acid molecule is operably linked to a promoter that drives expression in a cell.

5. A microbial cell having stably incorporated into its genome at least one nucleotide construct comprising a nucleic acid operably linked to a promoter that drives expression of said nucleic acid in said microbial cell, wherein said nucleic acid comprises the nucleotide sequence set forth in SEQ ID NO: 1, said nucleotide sequence encoding a protein inhibitory to Sclerotinia spp.

6. A microbial cell according to claim 5, wherein said nucleotide sequence encodes a protein comprising an amino acid sequence set forth in SEQ ID NO: 2.

7. A microbial cell according to claim 5, wherein the microbial cell is selected from a Pseudomonas sp.

8. A microbial cell according to claim 7, wherein the microbial cell is selected from a Pseudomonas chlororaphis strain.

9. A composition configured for inhibiting Sclerotinia infections of Brassica sp., said composition comprising a culture of microbial cells and a carrier, wherein said microbial cells comprise a nucleic acid molecule comprising a nucleotide sequence set forth in SEQ ID NO: 1, said nucleotide sequence encoding a protein comprising an amino acid sequence set forth in SEQ ID NO: 2.

10. A composition according to claim 9, wherein the culture of microbial cells comprises the microbial cells of claim 5.

11. A composition according to claim 9, wherein the culture of microbial cells is selected from a Pseudomonas sp.

12. A composition according to claim 9, wherein the culture of microbial cell is selected from a Pseudomonas chlororaphis strain.

13. A method for inhibiting Sclerotinia infections of Brassica plants, said method comprising coating Brassica seeds prior to sowing, with one of the microbial cells of claim 5 or the composition of claim 9.

14. A method for inhibiting Sclerotinia infections of Brassica plants, said method comprising applying to developing Brassica plants, one of the microbial cells of claim 5 or the composition of claim 9.


SEQUENCE LISTING (16 sequences)

SEQ ID NO: 1 - DNA, 906 bp, Pseudomonas chlororaphis strain PA23
atggatgacctggcggcgttcgccgtgctgatcgaggccggcagtttcaccctggccgcccagcaattgggttgcagcaaggggcaattgtccaagcgcatcagcgccctggaggcgcagttctcggtcgtgctgttgcagcgcaccacgcgccgcttgagcctgaccgcggcgggcgccgccttgttgccccaggcccaggcgctgctggtccaggtcgagcgggcgcgccaggcgctggcgcggctcaaggacgacatggccgggccggtgcggctgacggtgccggtgtccctgggggagacctttttcgagggcctgttgctggagttttcccggcagtatcccgaggtgcagatcgagctggagctgaacaacaactaccgcgacctgacccgggacggcttcgacctggcgatccgctcggaggtggccaacgaccagcggctggtggccaggccgctgctggcctggcacgagatgacctgcgccagcccggcttacctggagcaatatggcgagccgcagacgcccagggacctggccgagcatcgctgcctgctcaacagtcattacagcggtcgtgaggagtggctctatcaccagcagcacgagctgttgcgggtgcgggtgtcggggcccttcgccagcaatcactacaacctgctgaaaaaggccgcgctggtgggcgccgggatcgcccgactgccgtcctacctgttgcaggcggaactggccgatgggcgtttgcgcgggctgctgcgcgactaccagacccgcagcatgccgatgtacctggtgcacccgtatcagtatcagggcgggctgcccaggcgcacccaggtcctggccgattacctgatcggctggttcaagcgcagcggcgaagcgctggatcgcctctga

SEQ ID NO: 2 - protein, 301 aa, Pseudomonas chlororaphis strain PA23
MDDLAAFAVLIEAGSFTLAAQQLGCSKGQLSKRISALEAQFSVVLLQRTTRRLSLTAAGAALLPQAQALLVQVERARQALARLKDDMAGPVRLTVPVSLGETFFEGLLLEFSRQYPEVQIELELNNNYRDLTRDGFDLAIRSEVANDQRLVARPLLAWHEMTCASPAYLEQYGEPQTPRDLAEHRCLLNSHYSGREEWLYHQQHELLRVRVSGPFASNHYNLLKKAALVGAGIARLPSYLLQAELADGRLRGLLRDYQTRSMPMYLVHPYQYQGGLPRRTQVLADYLIGWFKRSGEALDRL

SEQ ID NO: 3 - DNA, 19 bp, Artificial sequence
  Plasmid pOT182
gggaaccggcttatagcca

SEQ ID NO: 4 - DNA, 21 bp, Artificial Sequence
  Plasmid pOT182-443 (XhoI)
atccagttgctggagcgtatt

SEQ ID NO: 5 - DNA, 20 bp, Artificial sequence
  Plasmid pCRR2.ITOPO
aagtacggggcgtaactgtc

SEQ ID NO: 6 - DNA, 19 bp, Artificial sequence
  Plasmid pUCP22
cggcctttttcagcaggtt

SEQ ID NO: 7 - DNA, 18 bp, Artificial sequence
  Plasmid pCR-phzAR
aatcctgccatccaactc

SEQ ID NO: 8 - DNA, 20 bp, Artificial sequence
  Plasmid pUCP22-phzR
aagttgttcgaaggggttca

SEQ ID NO: 9 - DNA, 20 bp, Artificial sequence
  Plasmid pCR-psrA
cttggcaatcctcctttttc

SEQ ID NO: 10 - DNA, 21 bp, Artificial sequence
  Plasmid pUCP22-ptrA
tagcttagcggatgtaagctg

SEQ ID NO: 11 - DNA, 24 bp, Artificial sequence
  Plasmid pPTRA-lacZ
ccggatccggtgacgccggtttca

SEQ ID NO: 12 - DNA, 20 bp, Artificial sequence
  Plasmid pPHZA-lacZ
cagcagggttttatccgaat

SEQ ID NO: 13 - DNA, 21 bp, Artificial sequence
  Plasmid pPHZR-lacZ
accatttcaacggggtctcac

SEQ ID NO: 14 - DNA, 20 bp, Artificial sequence
  Plasmid pPSRA-lacZ
tgactccatgtgacctccta

SEQ ID NO: 15 - DNA, 21 bp, Artificial sequence
  Plasmid pLP170
gatcctggaaaacgggaaagg

SEQ ID NO: 16 - DNA, 18 bp, Artificial sequence
  Plasmid pME3219
atgttaggaggtcacatg